(19) United States
(12) Patent Application Publication
(10) Pub. No.: US 2026/0199125 A1
(43) Pub. Date: Jul. 16, 2026

(54) Handheld Heating and Cooling Device

(71) Applicant: Luke Tipple, Pompano Beach, FL (US)

(72) Inventor: Luke Tipple, Pompano Beach, FL (US)

(21) Appl. No.: 19/334,276

(22) Filed: Sep. 19, 2025

Related U.S. Application Data
(63) Continuation-in-part of application No. 30/017,799, filed on Aug. 12, 2025.
(60) Provisional application No. 63/746,111, filed on Jan. 16, 2025.

(51) Int. Cl. A61F 7/00; F25B 21/04

(52) U.S. Cl. CPC A61F 7/007; F25B 21/04; A61F 2007/0036; A61F 2007/0075; A61F 2007/0077; A61F 2007/0087; A61F 2007/0095

(57) ABSTRACT

A handheld heating and cooling device is a device that enables regulation of body temperature by heating or cooling the user's hand using electrical power. The device includes an ergonomically shaped housing, a thermoelectric heat pump, a heat exchanger, a controller, a portable power source, and a thermo-conductive panel. The ergonomically shaped housing is a hollow structure large enough to retain the thermoelectric heat pump, the heat exchanger, the controller, and the power source. The thermo-conductive panel allows the warming or cooling of the user's body by allowing the thermal exchange between the thermoelectric heat pump and the user's palm. The thermoelectric heat pump generates the heating or cooling necessary to regulate the user's body temperature. The heat exchanger prevents damage to the internal components by removing heat waste from the thermoelectric heat pump during operation. The controller and the portable power source allow the mobile use of the device.

### FIELD OF THE INVENTION

The present invention relates generally to portable accessories and thermoelectric heating and cooling. More specifically, the present invention discloses a handheld portable device that utilizes electric power to heat and/or cool the user's body without the use of an external power source.

### BACKGROUND OF THE INVENTION

Nowadays, different wearable devices are available to help people regulate their body temperature. Some of the most popular ones are heating devices that help people stay warm such as thermal clothing, heating blankets, hand warmers, etc. In addition, cooling devices are available to help the user cool down such as cooling blankets, ice packs, etc. Most of these devices utilize chemical reactions or material's physical properties to warm or cool down the user's body. Thermoelectrical heating/cooling devices have been developed to help regulate the user's body temperature by using electric power. Most of these devices have been developed as portable devices with an integrated power system. However, these devices are often designed to either heat or cool down the user's body; few can do both in the same device. So, a handheld thermoelectric device able to warm and cool down the user's body is necessary.

Therefore, the objective of the present invention is to provide a handheld heating and cooling device able to warm up and cool down the user's body using electric power. The present invention implements a thermoelectric mechanism in a portable structure that can warm up or cool down the user's body temperature. Another objective of the present invention is to provide a handheld device with a portable power source that enables the mobile operation of the device. Further, another objective of the present invention is to provide a handheld device with an ergonomic design that is comfortable and safe to use. Additional features and benefits of the present invention are further discussed in the sections below.

### SUMMARY OF THE INVENTION

The present invention provides a handheld heating and cooling device. The present invention is a handheld device designed to warm up or cool down the user's core body temperature to provide relief from thermal stress or to increase physical performance. The human hand serves as a thermal conductor capable of affecting the entire body core temperature. With the press of a button, the user can select from a range of temperatures to suit the user's immediate needs. In the preferred embodiment, the present invention is designed to be highly portable and is small enough to fit in a handbag. In addition, the present invention includes a Universal Serial Bus (USB) rechargeable battery that can power the thermoelectric mechanism for around an hour of use (approximately 15 to 20 uses per charge). Further, the thermoelectric mechanism preferably utilizes a Peltier device encased in aluminum accompanied by a proprietary heat sink. This allows the efficient heating or cooling of the user's body by utilizing the same portable device. Furthermore, the present invention can include a ventilation mechanism that prevents damage to the electrical components during operation.

### BRIEF DESCRIPTION OF THE DRAWINGS

FIG. 1 is a top-front-left perspective view of the present invention.

FIG. 2 is a bottom-rear-right perspective view of the present invention.

FIG. 3 is a front view of the present invention.

FIG. 4 is a vertical cross-sectional view of the present invention taken along line 4-4 shown in FIG. 3.

FIG. 5 is a rear view of the present invention.

FIG. 6 is a right view of the present invention.

FIG. 7 is a box diagram showing the electrical connections and the electronic connections of the present invention, wherein the electrical connections are shown in solid lines, and wherein the electronic connections are shown in dashed lines.

### DETAIL DESCRIPTIONS OF THE INVENTION

All illustrations of the drawings are for the purpose of describing selected versions of the present invention and are not intended to limit the scope of the present invention.

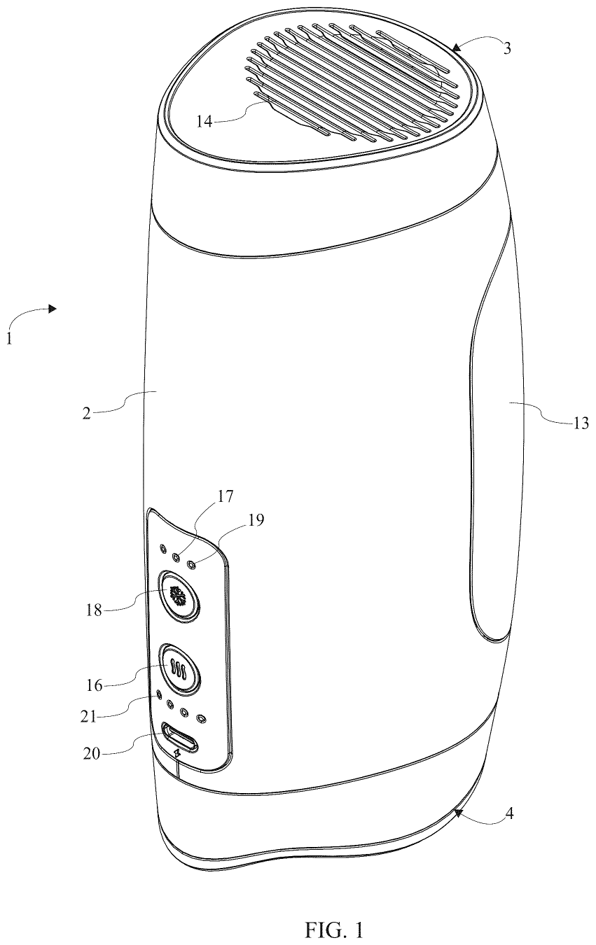

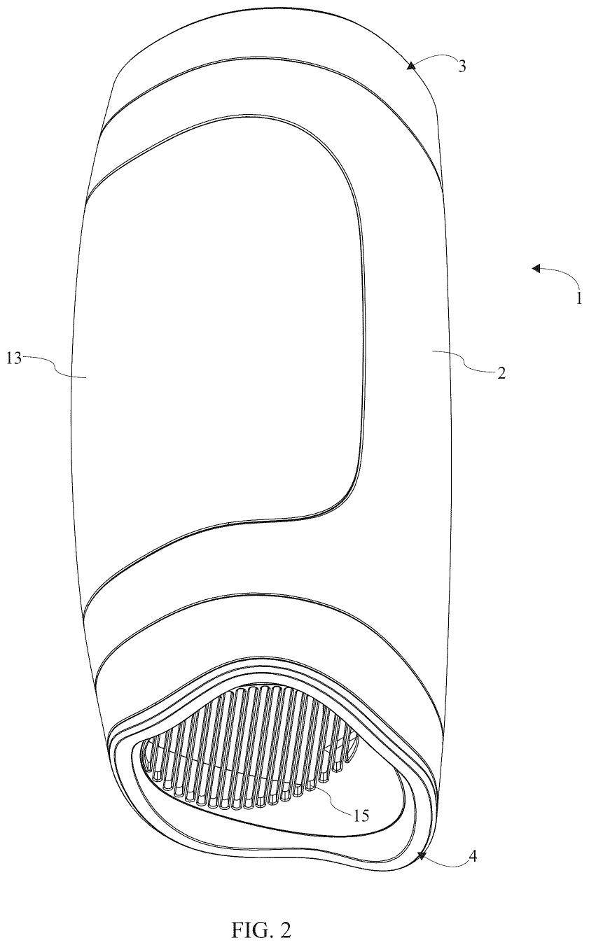

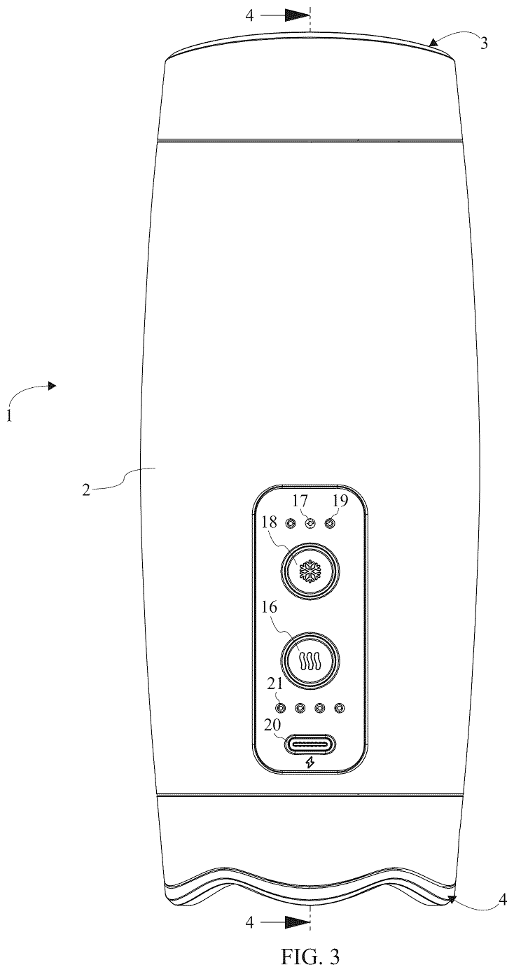

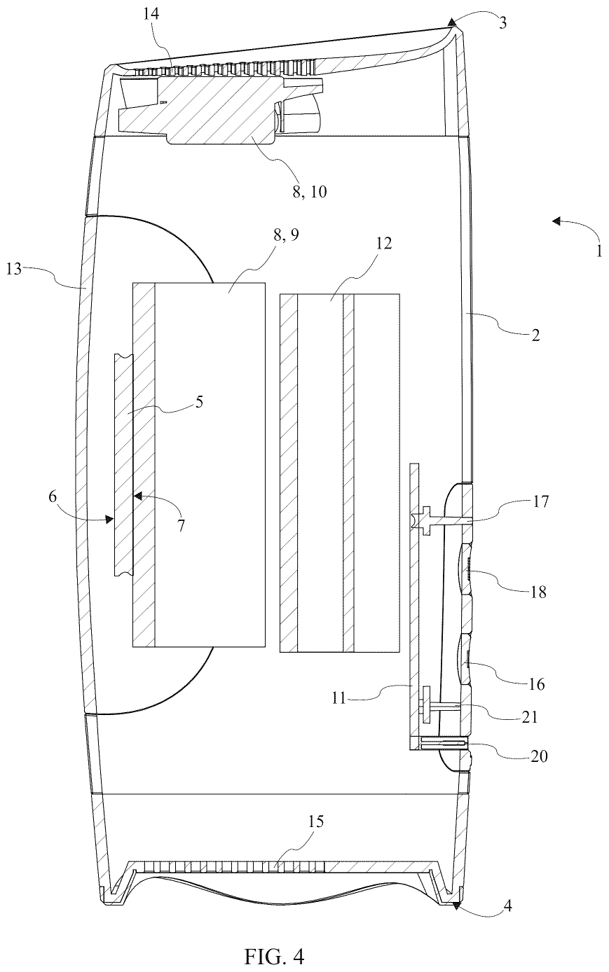

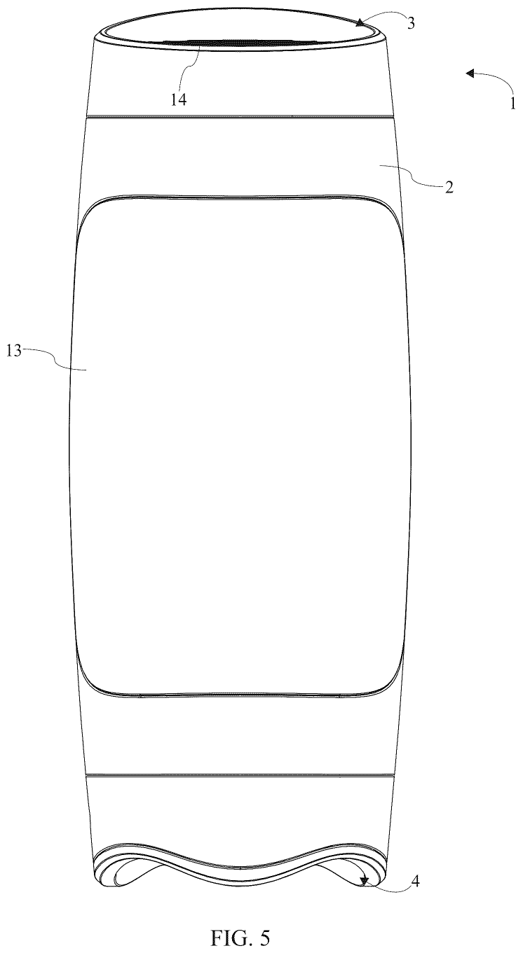

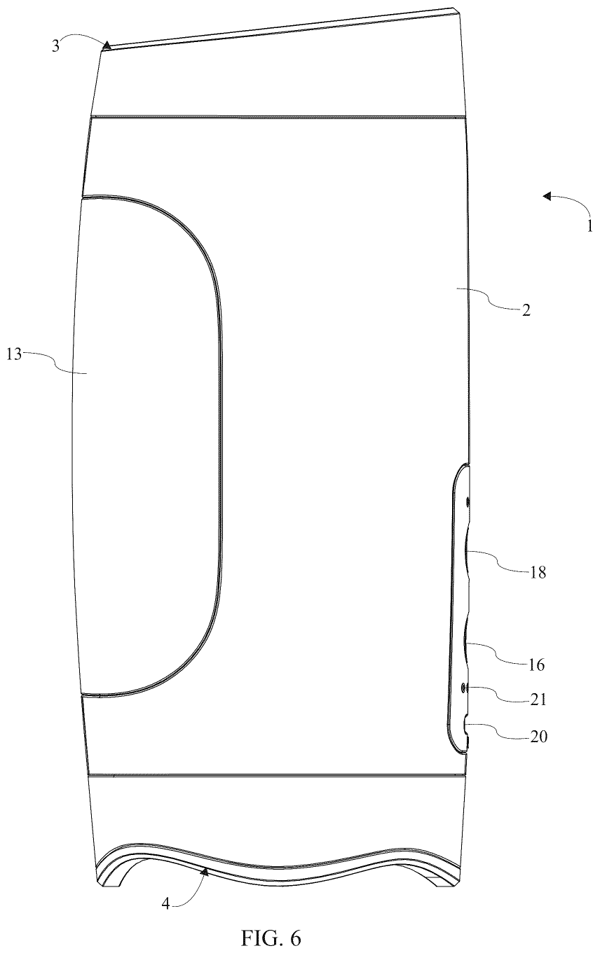

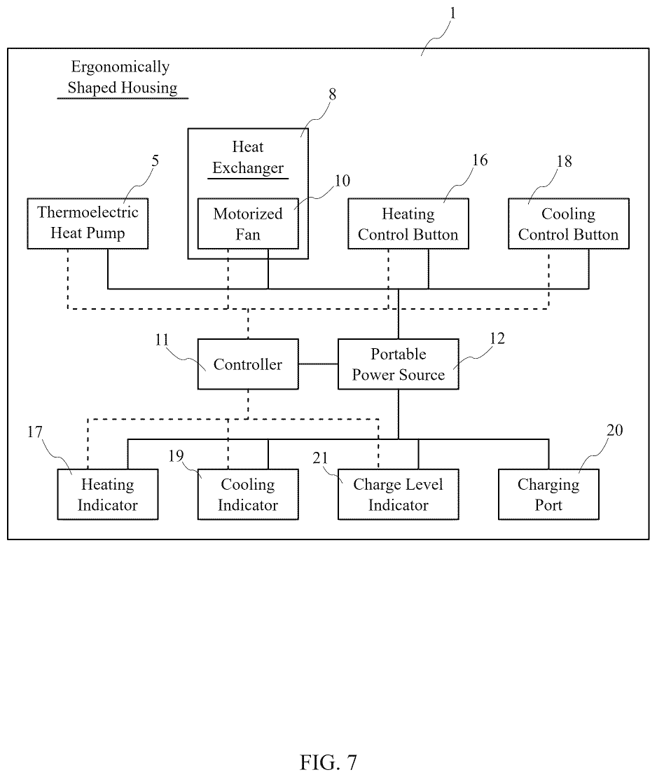

The present invention discloses a handheld heating and cooling device that enables the user to regulate the body temperature by heating or cooling the user's hand using the electrical power. In the preferred embodiment, the present invention comprises an ergonomically shaped housing 1, a thermoelectric heat pump 5, a heat exchanger 8, a controller 11, a portable power source 12, and a thermo-conductive panel 13, as can be seen in FIGS. 1 through 7. The ergonomically shaped housing 1 corresponds to an egg-shaped housing large enough to retain the thermoelectric heat pump 5, the heat exchanger 8, the controller 11, and the portable power source 12. The ergonomically shaped housing 1 also retains the thermo-conductive panel 13 in such a way that the ergonomically shaped housing 1 can be contact with the user's palm. The thermo-conductive panel 13 allows the warming or cooling of the user's body by allowing the thermal exchange between the thermoelectric heat pump 5 and the user's palm. The thermoelectric heat pump 5 is preferably a Peltier device that generates the heating or cooling necessary to regulate the user's body temperature. The heat exchanger 8 prevents damage to the internal components by removing heat waste from the thermoelectric heat pump 5 during operation. The controller 11 and the portable power source 12 allow the portable operation of the present invention.

The general configuration of the aforementioned components enables the user to effectively warm up or cool down using a single electrically powered device in a wide range of weather conditions. As can be seen in FIGS. 1 through 7, the ergonomically shaped housing 1 is designed as a portable egg-shaped structure large enough to fit on the user's hand. The overall shape of the ergonomically shaped housing 1 can vary but is designed to be comfortably gripped by the user's hand.

Further, the ergonomically shaped housing 1 can be sized to fit within a handbag, with an overall length being less than 170 millimeters (mm). In general, the ergonomically shaped housing 1 comprises a housing lateral wall 2, a first housing end 3, and a second housing end 4. The first housing end 3 and the second housing end 4 correspond to the terminal open ends of the ergonomically shaped housing 1, while the housing lateral wall 2 corresponds to the lateral portion of the ergonomically shaped housing 1. The first housing end 3 and the second housing end 4 are preferably open ends to allow airflow through the ergonomically shaped housing 1 to regulate the internal temperature of the electrical components during operation. The airflow through the ergonomically shaped housing 1 is directed longitudinally across the heat exchanger 8 to dissipate the heat waste from the heat exchanger 8.

Further, the housing lateral wall 2 provides a large surface that can be safely gripped by the user's hand. Due to the ergonomic egg-shaped design, the housing lateral wall 2 is not a straight wall. Instead, the housing lateral wall 2 has a curved structure that is thinner on the Z-axis and wider on the X-axis, as can be seen in FIGS. 1 through 7. In addition, two opposite surfaces of the ergonomically shaped housing 1 are designed in such a way that both are convex surfaces but one of the surfaces protrudes more than the other. The convex surface with greater curvature is preferably the palm-bracing surface when the user grips the ergonomically shaped housing 1. On the other hand, the convex surface with the smaller curvature is the finger-bracing surface when the user grips the ergonomically shaped housing 1. In other embodiments, the shape of the ergonomically shaped housing 1 can be altered to make the present invention more comfortable or practical to hold with one or more hands.

Further, in the preferred embodiment, the thermoelectric heat pump 5 utilizes the Peltier effect to generate the heating or cooling necessary to regulate the user's body. So, the thermoelectric heat pump 5 comprises a first thermal side 6 and a second thermal side 7 corresponding to two opposite large surfaces of the thermoelectric heat pump 5, as can be seen in FIGS. 1 through 7. Depending on the mode of operation, the first thermal side 6 and the second thermal side 7 can either heat up or cool down. The thermal mode of operation can be configured by controlling the polarity of the voltage delivered to the thermoelectric heat pump 5.

Generally, the present invention can be arranged as follows: the first housing end 3 is positioned opposite the second housing end 4 along the housing lateral wall 2 due to the overall elongated shape of the ergonomically shaped housing 1, as can be seen in FIGS. 1 through 7. The thermo-conductive panel 13 is integrated into the housing lateral wall 2 so that the thermo-conductive panel 13 can be in contact with the user's palm to allow the heat exchange between the user's palm and the thermoelectric heat pump 5. When the thermos-conductive panel 13 is in contact with the user's palm, the user's core body temperature can be altered via the direct thermal contact to the Arterio-Venous Anastomoses (AVAs) on the user's palm. Further, the thermoelectric heat pump 5, the heat exchanger 8, the controller 11, and the portable power source 12 are mounted within the housing lateral wall 2. This way, the electrical and electronic components of the present invention are protected by the ergonomically shaped housing 1. Further, the first thermal side 6 is preferably arranged to be in thermal communication with the thermo-conductive panel 13, while the second thermal side 7 is in thermal communication with the heat exchanger 8.

This arrangement allows two modes of operation for the present invention: heating mode and cooling mode. The thermo-conductive panel 13 can be made from anodized aluminum or other appropriate high-thermal conductivity materials to enhance the heat exchange through the thermo-conductive panel 13. When the present invention is engaged to cool the user's body down in a cooling mode, the thermoelectric heat pump 5 heats up the second thermal side 7 and the first thermal side 6 is cooled down, as can be seen in FIGS. 1 through 7. This results in the first thermal side 6 being the cold side and the second thermal side 7 being the hot side which cools down the thermo-conductive panel 13 to cool down the user's body. The thermo-conductive panel 13 can be cooled down to a temperature range between 12° C. and 16° C.

Alternatively, when the present invention is engaged to warm the user's body up in a heating mode, the thermoelectric heat pump 5 heats up the first thermal side 6 and the second thermal side 7 is cooled down. As a result, the first thermal side 6 is configured to be the hot side and the second thermal side 7 the cold side which warms up the thermo-conductive panel 13 to warm up the user's body. The thermo-conductive panel 13 can be heated up to a temperature range between 95° C. and 109° C. In other embodiments, different modes of operation can be implemented.

As previously discussed, this action can be performed by reversing the polarity of the voltage provided by the portable power source 12. Furthermore, the thermoelectric heat pump 5 and the heat exchanger 8 are electronically connected to the controller 11, as can be seen in FIGS. 1 through 7. In addition, the controller 11, the thermoelectric heat pump 5, and the heat exchanger 8 are electrically connected to the portable power source 12. Thus, the present invention can be powered by the portable power source 12 and controlled via the controller 11 so that the present invention can be used anytime anywhere.

As previously discussed, the second thermal side 7 is in thermal communication with the heat exchanger 8. This is so that the heat exchanger 8 can safely dissipate the heat waste during the cooling mode of the present invention. To do so, the heat exchanger 8 may comprise a heat sink 9 that absorbs the heat waste from the second thermal side 7 during the cooling mode, as can be seen in FIGS. 1 through 7. The heat sink 9 is mounted across the second thermal side 7 to increase the contact surface area between the heat sink 9 and the second thermal side 7. In addition, the heat sink 9 is in thermal communication with the second thermal side 7 to allow the heat exchange between the heat sink 9 and the second thermal side 7.

In some embodiments, the air flow through the ergonomically shaped housing 1 may not be enough to cool down the heat sink 9 during the operation of the present invention. To create greater airflow through the ergonomically shaped housing 1, the heat exchanger 8 may further comprise a motorized fan 10, as can be seen in FIGS. 1 through 7. In addition, the present invention may further comprise an inlet vent 14 and an outlet vent 15 that prevent small objects from entering the ergonomically shaped housing 1 and damaging the electronic and electrical components.

The inlet vent 14 is mounted into the first housing end 3 to secure the inlet vent 14 to the ergonomically shaped housing 1, as can be seen in FIGS. 1 through 7. On the other hand, the outlet vent 15 is mounted into the second housing end 4 to secure the outlet vent 15 to the ergonomically shaped housing 1. This way, both open ends of the ergonomically shaped housing 1 are covered with a corresponding vent to allow air flow through the ergonomically shaped housing 1 while preventing small objects from entering. Further, the motorized fan 10 is mounted adjacent to the inlet vent 14. This way, the air flow moves from the first housing end 3, through the ergonomically shaped housing 1, and out through the second housing end 4. Further, the motorized fan 10 is electronically connected to the controller 11 to automate the operation of the motorized fan 10. For example, the motorized fan 10 can be configured to operate during the cooling mode, and at a smaller scale during the heating mode. Furthermore, the motorized fan 10 is electrically connected to the portable power source 12 to receive the electrical power necessary for operation. In other embodiments, different mechanisms can be used for the heat exchanger 8.

The present invention is preferably designed so that the user can engage the desired mode of operation using a single button. To do so, the present invention may further comprise a heating control button 16, as can be seen in FIGS. 1 through 7. The heating control button 16 can be a tactile switch or other mechanical switch that allows the user to selectively engage the heating mode of operation. To do so, the heating control button 16 is integrated into the housing lateral panel, opposite the thermo-conductive panel 13. This way, the user can engage the heating control button 16 while holding the ergonomically shaped housing 1. The heating control button 16 is electronically connected to the controller 11 and electrically connected to the portable power source 12.

The heating control button 16 can be used to engage different heat intensities during the heating mode. The range of heating intensities can include three heating temperatures that are safe for the user's body, and the heating control button 16 is designed to cycle through the different heating intensities every time the user presses the heating control button 16. For example, at the initial press of the heating control button 16, the present invention is powered on and the first thermal side 6 is heated to a minimum heating temperature. When the heating control button 16 is pressed again, the first thermal side 6 is heated to an intermediate heating temperature. When the heating control button 16 is pressed again, the first thermal side 6 is heated to a maximum heating temperature. Finally, when the heating control button 16 is pressed again, present invention is powered off and the first thermal side 6 cools down.

To help the user keep track of the heating mode, the present invention may further comprise at least one heating indicator 17, as can be seen in FIGS. 1 through 7. The at least one heating indicator 17 is preferably a light indicator that is engaged when the heating mode is selected. To do so, the at least one heating indicator 17 is integrated into the housing lateral panel, adjacent to the heating control button 16. This way, the at least one heating indicator 17 is positioned adjacent to the heating control button 16 and remains visible when the user is gripping the housing lateral wall 2. Further, the at least one heating indicator 17 is electronically connected to the controller 11 and electrically connected to the portable power source 12. This way, the at least one heating indicator 17 is activated by the controller 11 when the heating mode is engaged using the electrical power provided by the portable power source 12. Furthermore, the at least one heating indicator 17 can be multiple heating indicators, each corresponding to a different heating intensity. In other embodiments, the at least one heating indicator 17 can be replaced with a different visual indicator.

Similar to the heating control button 16, the present invention may further comprise a cooling control button 18 used to selectively engage the cooling mode of the present invention, as can be seen in FIGS. 1 through 7. The cooling control button 18 can also be a tactile switch or other mechanical switch that allows the user to selectively engage the cooling mode of operation. To do so, the cooling control button 18 is integrated into the housing lateral panel, opposite the thermo-conductive panel 13. This way, the user can engage the cooling control button 18 while holding the ergonomically shaped housing 1. The cooling control button 18 is electronically connected to the controller 11 and electrically connected to the portable power source 12.

Like the heating control button 16, the cooling control button 18 can be used to engage different cooling intensities during the cooling mode. The range of cooling intensities can include three cooling temperatures that are safe for the user's body, and the cooling control button 18 is designed to cycle through the different cooling intensities every time the user presses the cooling control button 18. For example, at the initial press of the cooling control button 18, the present invention is powered on and the first thermal side 6 is cooled to a minimum cooling temperature. When the cooling control button 18 is pressed again, the first thermal side 6 is cooled to an intermediate cooling temperature. When the cooling control button 18 is pressed again, the first thermal side 6 is cooled to a maximum cooling temperature. Finally, when the cooling control button 18 is pressed again, present invention is powered off and the first thermal side 6 warms up.

To help the user keep track of the cooling mode, the present invention may further comprise at least one cooling indicator 19, as can be seen in FIGS. 1 through 7. Like the at least one heating indicator 17, the at least one cooling indicator 19 is preferably a light indicator that is engaged when the cooling mode is selected. To do so, the at least one cooling indicator 19 is integrated into the housing lateral panel, adjacent to the cooling control button 18. This way, the at least one cooling indicator 19 is positioned adjacent to the cooling control button 18 and remains visible when the user is gripping the housing lateral wall 2. Further, the at least one cooling indicator 19 is electronically connected to the controller 11 and electrically connected to the portable power source 12. This way, the at least one cooling indicator 19 is activated by the controller 11 when the cooling mode is engaged using the electrical power provided by the portable power source 12. Furthermore, the at least one cooling indicator 19 can be multiple cooling indicators, each corresponding to a different cooling intensity. In other embodiments, the at least one cooling indicator 19 can be replaced with a different visual indicator.

Further, in some embodiments, the present invention may further comprise a power indicator. Likewise, the power indicator can be a light indicator that is engaged when either the heating control button 16 or the cooling control button 18 is pressed. The power indicator remains active while either the heating or cooling mode of operation is engaged. The power indicator is integrated into the housing lateral wall 2, opposite the thermos-conductive panel. This way, the power indicator can be positioned adjacent to the at least one heating indicator 17 and the at least one cooling indicator 19. Further, the power indicator can be electronically connected to the controller 11 and electrically connected to the portable power source 12. In other embodiments, different visual indicators can be implemented to help the user monitor different operational features of the present invention.

As previously discussed, the portable power source 12 provides the electrical power necessary so that the present invention can be used on the go. As can be seen in FIGS. 1 through 7, the portable power source 12 is a portable battery that can be either recharged or replaced depending on the type of portable battery utilized. In the preferred embodiment, the portable power source 12 is a rechargeable battery, such as a lithium-ion rechargeable battery, with a capacity large enough to provide uninterrupted electrical power for several hours of use. However, other types of power storage devices can be implemented as necessary.

To enable the recharging of the portable power source 12, the present invention may further comprise a charging port 20, as can be seen in FIGS. 1 through 7. The charging port 20 is preferably a Universal Serial Bus (USB) charging port 20, but other technologies can be implemented that allow different charging methods, such as wireless charging. Further, the charging port 20 is integrated into the housing lateral wall 2, opposite the thermo-conductive panel 13. This way, the charging port 20 is positioned away from the thermo-conductive panel 13 and may be positioned adjacent to the heating control button 16 and the cooling control button 18. Furthermore, the charging port 20 is electrically connected to the portable power source 12 to allow the flow of electricity from an external power source to the portable power source 12 during the battery charging process.

Further, to help the user track the charge level of the portable power source 12, the present invention may further comprise at least one charge level indicator 21, as can be seen in FIGS. 1 through 7. Similar to the at least one heating indicator 17 and the at least one cooling indicator 19, the at least one charge level indicator 21 can be a light indicator that is activated when the present invention is turned on. The at least one charge level indicator 21 is integrated into the housing lateral wall 2, opposite the thermo-conductive panel 13, to keep the at least one charge level indicator 21 visible even when the user is gripping the housing lateral wall 2. Further, the at least one charge level indicator 21 is electronically connected to the controller 11 and electrically connected to the portable power source 12.

The at least one charge level indicator 21 can be provided as several charge level indicators to give the user a better idea of the current charge level of the portable power source 12. Depending on the charge level, the controller 11 activates a corresponding number of charge level indicators using the electrical power from the portable power source 12. For example, when all the charge level indicators are activated, the portable power source 12 is fully charged. When a single charge level indicator is activated, the portable power source 12 is almost empty, and the user should recharge the portable power source 12. In other embodiments, different types of visual indicators can be used for the at least one charge level indicator 21.

In general, the ergonomically shaped housing 1 is large enough to be easily stored in a small bag or pocket. However, the present invention can include other means of storage and transportation. For example the present invention may further comprise a strap and a strap eyelet. The strap eyelet is externally connected to the housing lateral wall 2, adjacent to the first housing end 3, to accommodate the strap. The strap is looped through the strap eyelet to secure the strap to the ergonomically shaped housing 1 so that the present invention can be carried from the strap. The strap can be designed as a removable strap or be permanently connected to the strap eyelet. In addition, the strap can include a length adjustment mechanism that allows the user to secure the strap to the desired object or body part. In other embodiments, different mechanisms can be implemented to secure the present invention to external objects for storage and transportation.

Although the invention has been explained in relation to its preferred embodiment, it is to be understood that many other possible modifications and variations can be made without departing from the spirit and scope of the invention.

What is claimed is:

1. A handheld heating and cooling device comprising:
an ergonomically shaped housing;
a thermoelectric heat pump;
a heat exchanger;
a controller;
a portable power source;
a thermo-conductive panel;
the ergonomically shaped housing comprising a housing lateral wall, a first housing end, and a second housing end;
the thermoelectric heat pump comprising a first thermal side and a second thermal side;
the first housing end being positioned opposite the second housing end along the housing lateral wall;
the thermo-conductive panel being integrated into the housing lateral wall;
the thermoelectric heat pump, the heat exchanger, the controller, and the portable power source being mounted within the housing lateral wall;
the first thermal side being in thermal communication with the thermo-conductive panel;
the second thermal side being in thermal communication with the heat exchanger;
the thermoelectric heat pump and the heat exchanger being electronically connected to the controller; and
the controller, the thermoelectric heat pump, and the heat exchanger being electrically connected to the portable power source.

2. The handheld heating and cooling device as claimed in claim 1 further comprising:
wherein the thermoelectric heat pump is configured into a cooling mode;
the first thermal side being the cold side; and
the second thermal side being the hot side.

3. The handheld heating and cooling device as claimed in claim 1 further comprising:
wherein the thermoelectric heat pump is configured into a heating mode;
the first thermal side being the hot side; and
the second thermal side being the cold side.

4. The handheld heating and cooling device as claimed in claim 1 further comprising:
the heat exchanger comprising a heat sink;
the heat sink being mounted across the second thermal side; and
the heat sink being in thermal communication with the second thermal side.

5. The handheld heating and cooling device as claimed in claim 4 further comprising:
an inlet vent;
an outlet vent;
the heat exchanger further comprising a motorized fan;
the inlet vent being mounted into the first housing end;
the outlet vent being mounted into the second housing end;
the motorized fan being mounted adjacent to the inlet vent;
the motorized fan being electronically connected to the controller; and
the motorized fan being electrically connected to the portable power source.

6. The handheld heating and cooling device as claimed in claim 1 further comprising:
a heating control button;
the heating control button being integrated into the housing lateral wall, opposite the thermo-conductive panel;
the heating control button being electronically connected to the controller; and
the heating control button being electrically connected to the portable power source.

7. The handheld heating and cooling device as claimed in claim 6 further comprising:
at least one heating indicator;
the at least one heating indicator being integrated into the housing lateral wall, adjacent to the heating control button;
the at least one heating indicator being electronically connected to the controller; and
the at least one heating indicator being electrically connected to the portable power source.

8. The handheld heating and cooling device as claimed in claim 1 further comprising:
a cooling control button;
the cooling control button being integrated into the housing lateral wall, opposite the thermo-conductive panel;
the cooling control button being electronically connected to the controller; and
the cooling control button being electrically connected to the portable power source.

9. The handheld heating and cooling device as claimed in claim 8 further comprising:
at least one cooling indicator;
the at least one cooling indicator being integrated into the housing lateral wall, adjacent to the cooling control button;
the at least one cooling indicator being electronically connected to the controller; and
the at least one cooling indicator being electrically connected to the portable power source.

10. The handheld heating and cooling device as claimed in claim 1 further comprising:
a charging port;
the charging port being integrated into the housing lateral wall, opposite the thermo-conductive panel; and
the charging port being electrically connected to the portable power source.

11. The handheld heating and cooling device as claimed in claim 1 further comprising:
at least one charge level indicator;
the at least one charge level indicator being integrated into the housing lateral wall, opposite the thermo-conductive panel;
the at least one charge level indicator being electronically connected to the controller; and
the at least one charge level indicator being electrically connected to the portable power source.

12. A handheld heating and cooling device comprising:
an ergonomically shaped housing;
a thermoelectric heat pump;
a heat exchanger;
a controller;
a portable power source;
a thermo-conductive panel;
the ergonomically shaped housing comprising a housing lateral wall, a first housing end, and a second housing end;
the thermoelectric heat pump comprising a first thermal side and a second thermal side;
the heat exchanger comprising a heat sink;
the first housing end being positioned opposite the second housing end along the housing lateral wall;
the thermo-conductive panel being integrated into the housing lateral wall;
the thermoelectric heat pump, the heat exchanger, the controller, and the portable power source being mounted within the housing lateral wall;
the first thermal side being in thermal communication with the thermo-conductive panel;
the heat sink being mounted across the second thermal side;
the heat sink being in thermal communication with the second thermal side;
the thermoelectric heat pump and the heat exchanger being electronically connected to the controller; and
the controller, the thermoelectric heat pump, and the heat exchanger being electrically connected to the portable power source.

13. The handheld heating and cooling device as claimed in claim 12 further comprising:
wherein the thermoelectric heat pump is configured into a cooling mode;
the first thermal side being the cold side; and
the second thermal side being the hot side.

14. The handheld heating and cooling device as claimed in claim 12 further comprising:
wherein the thermoelectric heat pump is configured into a heating mode;
the first thermal side being the hot side; and
the second thermal side being the cold side.

15. The handheld heating and cooling device as claimed in claim 12 further comprising:
an inlet vent;
an outlet vent;
the heat exchanger further comprising a motorized fan;
the inlet vent being mounted into the first housing end;
the outlet vent being mounted into the second housing end;
the motorized fan being mounted adjacent to the inlet vent;
the motorized fan being electronically connected to the controller; and
the motorized fan being electrically connected to the portable power source.

16. The handheld heating and cooling device as claimed in claim 12 further comprising:
a heating control button;
at least one heating indicator;
the heating control button being integrated into the housing lateral wall, opposite the thermo-conductive panel;
the heating control button being electronically connected to the controller;
the heating control button being electrically connected to the portable power source;
the at least one heating indicator being integrated into the housing lateral wall, adjacent to the heating control button;
the at least one heating indicator being electronically connected to the controller; and
the at least one heating indicator being electrically connected to the portable power source.

17. The handheld heating and cooling device as claimed in claim 12 further comprising:
a cooling control button;
at least one cooling indicator;
the cooling control button being integrated into the housing lateral wall, opposite the thermo-conductive panel;
the cooling control button being electronically connected to the controller;
the cooling control button being electrically connected to the portable power source;
the at least one cooling indicator being integrated into the housing lateral wall, adjacent to the cooling control button;
the at least one cooling indicator being electronically connected to the controller; and
the at least one cooling indicator being electrically connected to the portable power source.

18. The handheld heating and cooling device as claimed in claim 12 further comprising:
a charging port;
the charging port being integrated into the housing lateral wall, opposite the thermo-conductive panel; and
the charging port being electrically connected to the portable power source.

19. The handheld heating and cooling device as claimed in claim 12 further comprising:
at least one charge level indicator;
the at least one charge level indicator being integrated into the housing lateral wall, opposite the thermo-conductive panel;
the at least one charge level indicator being electronically connected to the controller; and
the at least one charge level indicator being electrically connected to the portable power source.
